(19) United States
(12) Patent Application Publication
(10) Pub. No.: US 2006/0147249 A1
(43) Pub. Date: Jul. 6, 2006

(54) Sealable sampling swab

(76) Inventor: Edward A. Fuller, Birmingham, MI (US)

Correspondence Address: HARNESS, DICKEY & PIERCE, P.L.C., P.O. BOX 828, BLOOMFIELD HILLS, MI, 48303, US

(21) Appl. No.: 11/026,901

(22) Filed: Dec. 30, 2004

(51) Int. Cl. A46B 11/00; A61M 35/00

(52) U.S. Cl. 401/118; 604/1

(57) ABSTRACT

A sealable swab is formed of an elongated stem having a swab collection material, such as a wad of cotton-like material or a spoon-shaped or a paddle-shaped end portion on one end. A tubular capsule enclosing and normally sealing the swab collection material within the capsule. The capsule is formed with an open top and a closed bottom within which a central opening is formed. The stem is slideably extended through the opening so that a portion of the stem with the swab collection material is arranged within the capsule. The open upper end of the capsule is closed by a cover that can be manually opened or closed with one-handed motion. When the cap is opened, the stem is moved endwise for moving the sample material outwardly of the capsule so that it may contact and accumulate material to be sampled, or, alternatively, may disperse sample material that was previously applied upon the swab. After the sample material has been collected or dispersed, the swab end may be retracted into the capsule and the capsule may be sealed by moving the cap into closed position over the open end of the capsule.

### FIELD OF THE INVENTION

This invention relates to a medical type swab which typically comprises an elongated stick or stem having a wad of cotton material or the like on one end for gathering sample materials that are to be laboratory tested. The present invention provides a system which encloses a swab within a protective container from which the swab can be easily and quickly withdrawn, using one hand, for collecting or for applying a sample and then replaced and sealed within the container.

### BACKGROUND OF INVENTION

It is common in conducting laboratory tests of samples of organic fluids and tissues to collect the samples on swabs which comprise elongated sticks having a wad of cotton-like material secured upon one end. The user of the swab holds the stick in one hand and dips or rubs the cotton end into the material from which a sample is taken. Then the sample may be tested on the swab, for example, for diagnostic purposes or for forensic investigations or for other biologically-related purposes. Conventional swabs may be easily contaminated both before their use and after collecting samples before the samples are tested. Hence, it is desirable to protect or seal the swabs both before and after the collection of a sample.

In the past, swabs have been packaged in a plastic or similar material bag or envelope, which requires tearing or otherwise opening the bag to remove the swabs for use. Then, after collection of the sample upon a swab, a container or bag has been used to store the swab and to protect the sample collected thereon from contamination. Frequently, in collecting a sample, the person using the swab needs to be able to use only one hand for removing the swab from its protective envelope or bag and for using the swab during collection of the sample and, then, to replace the swab within a protective covering. Thus, it is desirable to enable the user of a swab to uncover, manipulate, and re-cover a swab with one hand, leaving the other hand free for other uses, such as to hold a patient while a sample is collected on the swab or to hold an object, etc. This invention relates to a system by which a swab may be kept within a protective enclosure which can be easily opened, even with one hand, to free the swab for use and then closed after collection of the swab sample for protecting the swab sample against contamination.

Further, swabs are normally single-use items which are thrown away after use. Hence, for normal medical laboratory testing, it is essential to produce the swabs in their protective coverings at a minimal cost. This is particularly needed in a hospital or other medical or biological laboratory environment where large quantities of swabs are used.

Another use of swabs of the type which are utilized for collecting biological samples, is in applying material that is pre-deposited upon the swab, upon an article or object. For example, it would be useful to be able to use the swabs for applying cosmetics upon a person or for applying small drops of paint upon an object. For those purposes, it would be desirable to have a system for enclosing or encasing a swab upon which material has been pre-deposited and stored in a sanitary environment which protects the deposited material against contact with other objects or from evaporation or other losses. One example, such use would be the provision of a collection of swabs each carrying a different shade or color of lipstick so that the different lipstick samples may be applied and sampled by a user selecting one or more particular swabs having a color or shade desired by the user. Such a system, requires such a system to be inexpensive and yet adequate to seal and protect the material on the swab while still being easily and quickly accessible by the user.

### SUMMARY OF THE INVENTION

This invention contemplates a combined swab and protective container within which the tip portion of the swab is normally enclosed within a sealed capsule or housing, from which the tip may be momentarily extended and exposed for collecting or for dispersing sample materials, following which the swab may be retracted for protecting the tip and its contents against contamination or loss. The system involves a conventional swab which comprises a long stick or stem having a cotton-like tip portion for gathering or dispersing sample materials and a capsule or housing which temporarily encloses the swab tip portion.

The capsule is preferably formed of an elongated tube having a top open end and a bottom closed end. A hole or passageway is formed in the bottom end through which the stick or stem of the swab is slideably inserted. The tip end of the swab is normally positioned within the container and may be moved out of the open end of the container for exposing the tip for use and for retracting the tip into the container for storage.

The area around the hole through which the stick extends is preferably sealed with a suitable grommet or washer. This seals the stem around the periphery of the hole. A cap is hingedly connected to the upper end of the capsule and is secured in closed position over the capsule upper end by a suitable latch or flange arrangement for sealing the swab tip within the capsule.

With this system, the user may hold the stick and the capsule in one hand, push the cap so that it hingedly moves into its open position to expose the open end of the capsule. Then the user may push the stick upwardly such as by placing the free end of the stick against a table or other solid object while holding the capsule in hone hand. Thus, the tip of the swab will extend outwardly of the open end of the capsule. When the swab tip is exposed, the capsule remains upon the swab stem. Next, the tip may be contacted against an object from which a sample is to be obtained or upon which a sample is to be deposited. The object may be human body tissue or liquids or other biological tissues or materials, for example. As is conventional, a small amount of sample material will collect upon or be deposited from the swab tip. Then, the user may pull the stem rearwardly, relative to the capsule, or move the capsule forwardly to again encase the tip. Upon retracting the tip within the capsule, the user can swing the cover closed to seal the capsule with the tip and sample therein. This manipulation can be accomplished with one hand.

As mentioned, the system may be used as an applicator, for dispersing small quantities of materials that have been collected earlier on the tip of the swab. This is useful in dispensing cosmetic materials or small dabs of paint or coloring material and the like with a swab. For example, a series of swabs may be provided with samples of different shades or colors of lipstick or other cosmetics with each swab encased within its own capsule for protecting the sample until use. At the time of use, the user may flip the cover to open the end of the capsule and to slide the stick relative to the capsule for exposing the tip beyond the open end of the capsule. Then the contents on the tip may be applied to an object to be coated, which could be on the lips of a person or, the case of a dab of paint, upon an automotive vehicle or household furniture or other structures to be painted. After the application of the material from the swab, the capsule or the stem may be relatively moved to again enclose the tip of the swab within the capsule. Then the cap may be replaced into its covering position. The swab may be provided for single use and discarded after a single use or, alternatively, depending upon the materials collected or dispersed, may be reused with the material remaining on the swab protected against contamination during the storage or non-use time.

One object of this invention is to provide a simple, inexpensive system for protecting a swab against contamination before the swab is used for collecting test samples and after it has collected the test samples.

Another object of this invention is to provide a simple, combination swab and protective enclosure which can be easily operated, by one-hand manipulation to expose the swab for either collecting samples or dispersing sample collected thereon, protecting the materials upon the swab, and dispersing the materials from the swab when desired.

A further object of this invention is to provide a conventional medical-type of swab with a protective enclosure from which the swab may be extended or retracted for collecting, storing, and dispersing samples applied upon the swab.

Still a further object of this invention is to provide a highly inexpensive, simple enclosure within which the tip of the swab may be enclosed and, thereby protected against contamination or unwanted contact with other objects. The swab may be easily and quickly exposed or retracted when desired.

These and other objects and advantages of this invention will become apparent upon reading the following description, of which the attached drawings form a part.

### DESCRIPTION OF THE DRAWINGS

FIG. 1 illustrates a perspective view of the swab and container assembly.

FIG. 2 is an enlarged, cross-sectional view of the container and the upper portion of the swab with the swab tip enclosed within the capsule container.

FIG. 3 is a view similar to FIG. 2, illustrating the cap of the capsule in open position and the swab extending through the open end for collecting or dispersing material therefrom.

FIG. 4 illustrates, in cross-section, the disassembled parts of the container.

FIG. 5 schematically illustrates one form of swab having a paddle type end for collecting samples of solid materials.

FIG. 6 is another form of swab utilizing a spoon-shaped end for collecting solid materials.

FIG. 7 is a modification in which a lug for securing a tag is formed integrally with the capsule.

### DETAILED DESCRIPTION OF THE PREFERRED EMBODIMENTS

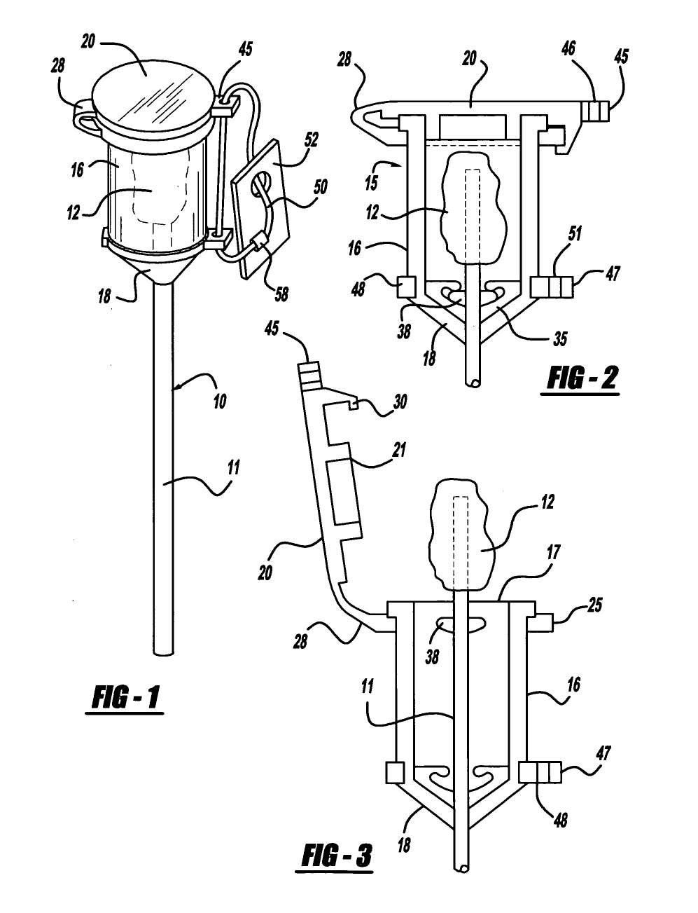

Referring to the drawings, FIG. 1 illustrates a conventional swab 10 formed of an elongated stick or stem 11 and a tip 12 formed of a wad of cotton-like material wound or packed upon the end of the stick.

The upper-end portion of the swab stick, with the tip material, is normally enclosed within a container 15, as illustrated in FIG. 2. The container is formed of a capsule or housing 16 which is tubular in shape. The capsule has an upper, open end 17 and a closed lower end 18. A central hole or passageway 19 is formed in the lower end for slideably receiving the swab stick.

A cap 20 is provided to temporarily cover the open end 17 of the capsule. The cap is provided with an inner annular flange 21 which fits within the open end of the capsule. Preferably the flange fits sufficiently snuggly to seal the open end of the capsule when the cap is applied in its closure position.

An edge bead or flange 23 is formed integrally with the upper end of the capsule. Thus, the cap is formed with a separate cap ring 25 that fits around the capsule, against the bead. A rabbit groove 26 formed in the interior of the ring seats the ring against the bead or flange.

An integral hinge portion 28, formed of a resiliently-bendable plastic material hingedly interconnects the cap with the cap ring 25 and that, in turn, connects the cap to the capsule. Thus, the cap may be flipped open by holding the capsule in one hand and pushing the cap with the thumb of the user to swing the cap into an open position, as illustrated, for example, in FIG. 3.

In order to hold the cap in closed position, it is provided with a latch tongue or lug 30, which is a flexible portion formed on the cap and extending radially outwardly of the cap for engaging around the cap ring. By forming the cap, with its cap ring of a suitable plastic material which has some slight resiliency, the latch tongue is likewise sufficiently resilient to slip over and grasp the ring for locking the cap over the open end of the capsule, as shown in FIG. 2.

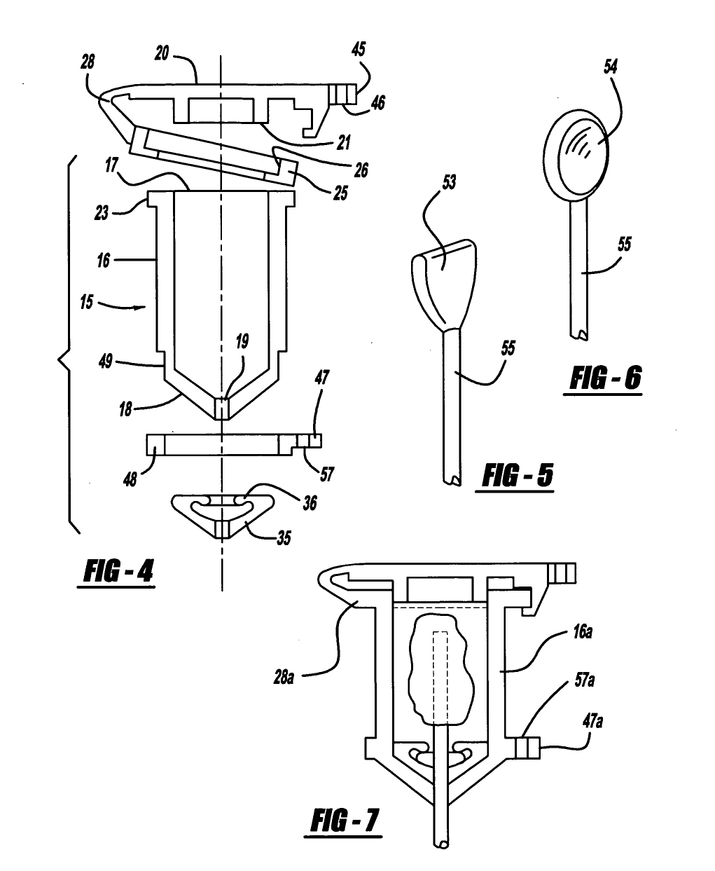

When the swab is inserted in the capsule for the first time, the cap would be opened and the swab stick slid through the hole 19 in the lower end 18 of the capsule. In order to seal the upper end of the swab within the container, a grommet 35 is arranged within the capsule. The grommet may be formed of a rubber-like material and be shaped in a roughly truncated triangular shape, inverted, with a central opening within which a radially inwardly extending grip flange 36 is formed (see FIG. 4). A washer 38 is positioned upon the stick and preferably is shaped to fit within the center portion of the grommet as illustrated in FIG. 2. However, other washers or grommet shapes may be used for sealing together the lower end of the capsule and the swab stick.

In use, it is desirable to have a way of identifying the individual swabs when they are stored with material collected upon them. For that purpose, a lug 45 is formed on the cap. The lug has a hole 46 formed therein. A corresponding lug 47 is formed on a lower ring 48 which surrounds the lower end of the capsule and is arranged within an open groove or annular open channel formation 49 on the capsule.

To identify a particular enclosed swab, a string or wire 50 may be passed through the hole 46 on the lug 45 and a hole 51 formed in the lug 47 on the lower ring. The string may be passed through one or more holes 53 in a tag 52 upon which information can be written to identify the particular swab contents.

FIG. 7 illustrates a modification in which a lug 47a is formed integrally with or on a ring slipped over the capsule 16a. The lug may be molded at the lower part of the capsule 16a or elsewhere on the capsule. A hole 57a is formed in the lug for tying the string of a tag which may contain information relevant to the sample. Also, the cap hinge 28a may be integral with the capsule.

In operation, the combined swab and container are kept together and may be sterilized, for laboratory test purposes, to protect the tip end of the swab. The user of the swab, may hold the swab in one hand, by holding the container and possibly part of the stem or stick and can flip the cover open with a movement of the thumb of the hand holding the unit. Then, the stick may be pushed longitudinally, such as by pressing it against a solid object, as for example, a table top, to move the tip upwardly and out of the container or, conversely, the stick may be held against some solid object and the hand-held container slid downwardly for relative movement of the tip outwardly of the container. Then, the swab may be applied to the material from which a sample is to be obtained. Next, the container and stick are relatively manipulated to retract the tip material back into the interior of the container. Then the cover may be swung back into closed position. In that way, the sample can be easily collected and once collected is protected against contamination until such time as it is transported to the place where tests are to be conducted and at least some of the contents are to be dispersed from the swab tip for that purpose.

Where the swab is to be used as an applicator, the material to be dispersed can be applied upon the swab tip and then the swab tip can be withdrawn until it is positioned within the container and the cap is closed to protect the material against the contamination or contact with other materials or objects. In use, the cap is flipped open by simply holding the container in one hand and pushing the cap with the thumb, or with the opposite hand, and manipulating the capsule and stick for exposing the tip of the swab outwardly of the capsule. After the material on the swab is applied upon the object desired, the tip can be retracted within the container for further use or for being discarded, as appropriate.

The particular tip used on the swab may vary depending upon the use of the swab. Thus, for collecting samples of solid or semi-solid materials with the tip of the swab, instead of being formed with a wad of cotton-like material, may have a paddle shape end 53 (see FIG. 5) or a spoon shape 54 formed upon a stem 55 (see FIG. 6). Also, although not illustrated, the end could be in the form of a brush with bristles which could be particularly useful in applying small amounts of paint or similar coatings and protecting the paint or coatings during storage or non-use.

The details of the construction of the cap, the hinge, and the interconnections between the cap and the capsule, as well as the structure and shape of the grommet, washer and other similar sealing devices may be varied within the scope of this invention. Such variations would be selected by those skilled in the art depending upon availability. Thus, the descriptions and illustrations of these various parts should be construed as being merely illustrative of an operative embodiment of this invention and not in a strictly limiting sense. Hence, having fully described an operative embodiment of this invention, the following claims should be construed as illustrative of the invention.

1. A sealable sampling swab comprising:
a hollow, tubular capsule having an upper open end and a lower closed end with a central hole formed in the lower closed end;
an elongated stem slideably extending through the central hole into the capsule and having an upper portion normally arranged within the capsule;
a sampling member formed on the upper end portion for holding material to be sampled, said member being normally arranged within the capsule;
said sampling member being extendable outwardly of the open upper end of the capsule by sliding the stem endwise, upwardly further into the capsule so that its upper end portion, with the sampling member is exposed outwardly beyond the upper open end of the capsule;
a cap normally closing the capsule open upper end, with said cap being manually removable for extending the sampling member outwardly of the capsule open end and being manually closeable for sealing the sampling member within the capsule;
whereby the sampling member may be manually moved from the capsule for obtaining a sample thereon or for delivery of a sample therefrom and otherwise is contained within, and substantially sealed within, the capsule.

2. A sealable sampling swab as defined in claim 1 and including:
a resilient grommet arranged within, and upon the lower end of the capsule, with the grommet having a hole aligned with the hole in the lower end;
said stem being fitted in, and being slideably extendable and retractable through said grommet for sealing the stem to the lower end of the capsule.

3. A slideable sampling swab as defined in claim 2, and including a ring-like washer mounted upon the stem portion within the capsule, said washer normally being in sealing contact with said grommet when the upper portion of the stem, with the sampling member, is wholly contained within the capsule.

4. A sealable sampling swab as defined in claim 2, and including a hinge connecting the cap to the upper end of the capsule for swinging the cap into and out of covering position relative to the open upper end of the capsule.

5. A sealable sampling swab as defined in claim 4, and including a depending flange formed on the cap for engaging the upper portion of the capsule for temporarily locking the cup in position for covering the open end of the capsule, for thereby sealing the capsule when the sampling swab is contained therein.

6. A sealable sampling swab as defined in claim 5, and said flange being sized and shaped to fit into the upper end portion of the capsule for engaging against, and sealing against, the interior circumferential surface of the wall which forms the capsule.

7. (canceled)

8. A sealable sampling swab comprising:
an elongated stem having an upper end portion with a sampling member formed thereon, and a tube-like capsule within which the stem upper end portion and sampling member are normally positioned;
said capsule having an open upper end and a closed lower end, with a passageway formed in the closed lower end;
said stem extending through the passageway and being manually slideable lengthwise through the passageway so that the stem upper end portion is extended outwardly of the open upper end of the capsule and is manually retractable for positioning the sampling member within the capsule;
a cap fitted over, and covering the upper open end of the capsule and being manually removable and replaceable therefrom for uncovering the capsule for movement of the swab sampling member out of the capsule for delivering or receiving a sample and being replaceable for sealing the swab member within the capsule.

9. A sealable sampling swab as defined in claim 8, and including a hinged member connecting said cap to the capsule with said cap being pivotal relative to the upper end of the capsule into position for covering and for uncovering the open end of the capsule.

10. A sealable sampling swab as defined in claim 9, and including cooperating latch members formed on the cap and capsule for releaseably interconnecting and holding the cap in closed position over the open end of the capsule for sealing the swab member within the capsule.

11. A sealable sampling swab as defined in claim 10, and including a resilient, ring-like grommet having a central passageway arranged within the capsule with said passageway being in alignment with the passageway formed in the capsule closed lower end, and said stem extending through the aligned passageways for sealing the stem upper portion and sampling member within the capsule relative to the lower end of the capsule.

12. A sealable sampling swab as defined in claim 10, and including a ring-like washer mounted upon the stem portion that is normally arranged within the capsule for engagement with, and sealing against, said grommet when the sealing member is arranged within the capsule.

13. A sealable sampling swab as defined in claim 8, and said cap having a lug formed thereon and with a corresponding lug formed on the capsule; an opening formed in each lug and the lug openings being relatively aligned;
whereby a cord-like element may be extended through both lugs and through a tag for locking the cap against opening relative to the upper end of the capsule, while simultaneously providing a tag for the placement of indicia identifying the sample contained upon the sampling member.

14. A method for collecting or dispersing samples of material and substantially sealing the samples against the atmosphere when such samples are accumulated after collection or before dispersement, including the steps of:
providing an elongated tubular capsule having an open upper end and a closed lower end and forming a central hole in the closed lower end;
inserting an elongated stem through said hole so that the stem has an upper portion arranged within the capsule and an elongated lower portion extending outwardly of the lower portion of the capsule;
providing a sampling collection and holding member upon the stem portion which is to be positioned within the capsule, before inserting the stem into and through the capsule;
providing a cap which normally covers and seals the upper end of the capsule, but which is manually removable for opening the upper end of the capsule;
moving the stem endwise to extend the stem portion and the sampling member formed thereon outwardly of the open end of the capsule;
collecting sample material on, or distributing sample material from, the sampling member;
retracting the stem portion endwise for repositioning the sampling member within the capsule and closing the cap for sealing the sample within the capsule;

15. A method as defined in claim 14, and including providing a seal between the stem and the hollow in lower end of the capsule;

16. A method as defined in claim 14, and including providing a hinged portion between the cap and the capsule for swingably holding the cap over the open end of the capsule; and providing a latch member for sealing the cap to the open end of the capsule when the cap is in position to cover the open end of the capsule;
whereby the cap may be swung into an open position relative to the capsule for extending the sampling material from the capsule and the cap may be repositioned into sealing engagement with the capsule by single-handed movement of the user's hand.

17. A method as defined in claim 16 and including assembling a substantial number of swabs and corresponding capsules, and applying upon the sampling material of each swab a different cosmetic substance, such as different color lipstick or paint-like coating samples; storing the swabs within their respective capsules; selecting one or more of the swabs from the number of swabs and dispersing the sample material from the selected swabs upon a person for testing the selected materials by that person.
